(19) United States
(12) Patent Application Publication
(10) Pub. No.: US 2005/0176019 A1
(43) Pub. Date: Aug. 11, 2005

(54) Method for identification of the location of mutations in whole genomes

(76) Inventors: Bruce A. Beutel, Lake Forest, IL (US); Claude G. Lerner, Highland Park, IL (US); Stephan J. Kakavas, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/775,409

(22) Filed: Feb. 10, 2004

(51) Int. Cl. C12Q 1/68; G06F 19/00; G01N 33/48; G01N 33/50; C12N 15/74

(52) U.S. Cl. 435/6; 435/471; 702/20

(57) ABSTRACT

This invention provides a method for identifying the precise locations of mutations in whole genomes using restriction enzymes and transformation frequency data.

### FIELD OF THE INVENTION

The invention relates generally to the field of mutations in whole genomes and their localization. Specifically, the invention relates to a method of identification of mutations using restriction enzymes and transformation frequency data.

### BACKGROUND OF THE INVENTION

The following background information is not admitted to be prior art to the claimed subject matter, but is provided to aid the understanding of the reader.

The ability to detect mutations in genomic (chromosomal) DNA is important for the identification of genetic determinants of particular phenotypes, for example the presence of inherited diseases, and in the case of bacteria, the determination of resistance to certain antibacterial compounds.

Antibacterial activity is the ability of a compound to prevent growth of bacteria. Some bacteria that can grow in the presence of the compound can be isolated at low frequencies by exposing sufficient number of cells to the compound and selecting those cells that are capable of growing in the presence of the compound. These strains are characterized as being phenotypically resistant to the compound. Resistant strains typically have one or more point mutations in the genomic DNA, which confers the resistance phenotype. For certain bacterial species, genomic DNA from a resistant bacterial strain can be used to transform a susceptible cell into a resistant cell by incorporating a segment of the mutant DNA into the chromosome of the susceptible cell.

Identification of the location of resistance mutations in bacterial genomes provides useful information about the mechanism of resistance. This can help explain clinical resistance in various settings including new mechanisms of emerging resistance to existing marketed drugs, as well as newly approved drugs. Identification of the location of resistance mutations in bacterial genomes is also important as a method for the discovery of targets for novel antibacterial agents with unknown mechanisms of action.

Several methods are available for determining where point mutations are located along bacterial genomes.

Classical genetic mapping requires a set of tester strains each with a mutation, or insertion, that confer selectable phenotypes (such as resistance to an antibiotic) at different known locations in the chromosome. DNA from the resistant strain is introduced into each tester strain and the cells are plated under conditions that require both mutations to be present for cell growth. When the locations of the reference mutation and the resistance mutations are close, the frequency of obtaining cells containing both mutations is higher than when the two mutations are far from each other. In this method the position of the resistance mutation is determined relative to known genetic markers. This method is slow, low throughput and yields a very low-resolution estimate of the location of the mutation in the genome (Bacterial and Bacteriophage Genetics, Fourth Ed. (2000, E. A. Birge, Springer-Verlag, N.Y.).

Another method involves cloning of resistance mutations by preparation of a library of DNA from a resistant strain in a plasmid vector that can replicate in the organism of interest. The library of genomic DNA from the resistant strain is then introduced into susceptible cells of the same species by transformation or electroporation. Resistant transformants are selected by the same means used to select the resistant mutant. The plasmid is isolated from the cells and the cloned DNA sequenced to identify the genes it contains. The sequence of the same region of the susceptible parent strain's genome is sequenced to identify nucleotide difference(s) in the resistant and susceptible strains. Problems with this method however, include the need for the resistance mutation to be dominant over the un-mutated version. Also, in certain cases increasing the copy number of some genes could confer drug resistance. In such cases the actual mutation that confers resistance to the antibacterial agent would not necessarily be identified. Furthermore, plasmid libraries can be difficult to construct and can be biased with certain sequences represented infrequently, or not at all, therefore making the resistance mutation not even present in the library.

A similar method involves cloning of the resistant organism DNA into bacteriophage vectors such as lambda, which are then used to infect host strains that can be plated and pooled. The cloned DNA is amplified from the pool by the polymerase chain reaction (PCR; Saiki, R. K., et al., Science Vol. 239(4839), pages 487 to 491 (1988)) and used to transform a susceptible strain into a resistant one. The positive lambda clones are sequenced to identify the regions of DNA contained in the clone. The corresponding region of the resistant mutant and susceptible parent are sequenced using PCR products as templates and the sequences are compared to identify the exact location of the mutation (Adrian, P. V. et al., Antimicrob. Agents Chemother., Vol. 44, pages 732 to 738 (2000)). As with the plasmid libraries, the lambda libraries can be (1) difficult to construct, and (2) biased with certain sequences represented infrequently, or not at all, therefore making the resistance mutation not even present in the library.

Another method to identify resistance mutations involves mutagenized PCR products covering all regions of the chromosome (Belanger, A. E. et al., Antimicrob. Agents Chemother. Vol 46, pages 2507 to 2512 (2002)). The method involves designing and synthesizing oligonucleotide primers to use in error prone PCR reactions to amplify the entire bacterial genome in 521 specific sections of approximately 4 kb in length. The mutagenized PCR products are pooled in groups and tested in transformation reactions with the sensitive strain to see which pool of mutagenized PCR products confers resistance to the compound. Individual PCR products from positive pools are then tested to determine which product contains a mutagenized species that confers resistance at high frequency. Poor representation, thus underestimation, of certain types of resistance mutations in the pools, makes this method less than optimal, in addition to being time and labor consuming.

Other more general non-phenotypic methods focus on identifying a physical mismatch in DNA heteroduplexes formed between mutated and non-mutated samples, based on physico-chemical differences between the duplexes. In this category, the GIRAFF (Genomic Identity Review by Annealing of Fractioned Fragments; Sokurenko, E. V. et al., Trends in Microbiology, Vol 9, pages 522 to 525 (2001)) and the MutS-RDA methods (Gotoh, K. et al., Biochem Biophys Res Commun., Vol 268, pages 535 to 540 (2000)) have been used with certain success. Such methods provide information about the physical location of nucleotide sequence differences in bacterial chromosomes. Multiple sequence differences are often found of which only a subset are related to the mutant phenotype. Therefore such methods are less than optimal since additional experiments must be performed to identify which nucleotide sequence difference is responsible for the mutant phenotype. In addition, these methods are less than optimal since they are low throughput, time and labor consuming.

Therefore, there is the need for a method for determining the locus of mutations of particular phenotypes in genomes, that: (a) is rapid, (b) is efficient and, (c) yields a high resolution estimate of the mutation locus.

### BRIEF SUMMARY OF THE INVENTION

The present invention relates to a method for identifying the location of a mutation in genomes. The method comprises the steps of: a) isolating genomic DNA from an organism having a mutated phenotype, b) digesting samples of isolated DNA with a set of restriction enzymes; c) transforming a non-mutant host strain with the digested DNA fragments; d) assessing the frequency with which the host strain is transformed to acquire the mutant phenotype, and e) identifying the location of the mutation by determining the regions of the genome restriction site map, derived from available genomic sequence data, that best fit the transformation frequency data.

The present invention also relates to a method for identifying the precise locus and identity of a mutation in the genome of a mutated organism. Said method comprises the steps of: a) isolating genomic DNA from an organism having a mutated phenotype, b) digesting samples of isolated DNA with a set of restriction enzymes; c) transforming a non-mutant host strain with the digested DNA fragments; d) assessing the frequency with which the host strain is transformed to acquire the mutant phenotype, and e) identifying the location of the mutation, and further comprising the steps off) amplifying the location by polymerase chain reaction using DNA of the mutant as a template, g) testing the amplified location for the ability to transform non-mutant host cells, h) sequencing the amplified location that transforms with high frequency and i) comparing said sequence to the sequence of the parent strain to precisely identify the locus and identity of the mutation.

The present invention also relates to a computerized method for identifying the location of a mutation in the genome of particular organisms using a computer program. The method comprises the steps of: a) inputting enzyme transformation data into a computer, wherein said enzyme transformation data comprises the results of frequency of transformation of non-mutated host organism after introduction of DNA fragments from a mutated organism, wherein said DNA fragments have been digested by known restriction enzymes, b) inputting known map of restriction enzyme cleavage sites into said computer, c) inputting a group of variables that affect frequency of transformation into said computer, d) correlating inputs of steps (a), (b), and (c) to genome coordinate through said computer program, wherein said computer program scans genome sequence to identify locations of restriction enzyme cleavage sites in the genome that best fit the transformation frequency data, and e) comparing the transformation frequency data with the genome restriction enzyme cleavage map to identify the location of the mutation.

### BRIEF DESCRIPTION OF DRAWINGS

The present invention will be further described with respect to the drawings wherein:

FIG. 1 graphically represents the dependence of transformation frequency on the distance of a mutation from the end of a fragment using PCR products of constant length containing the ciprofloxacin resistance mutation of the H. influenzae gyrA at different locations along the length of the fragment;

FIG. 2 graphically represents the dependence of transformation frequency on the length of restriction fragments using the engineered Abbott A-583 resistant fadL H. influenzae strain FLUSKO;

FIG. 3 graphically represents the map of restriction enzyme cleavage sites in the region of the known rifampicin resistance mutation in the B. subtilis rpoB gene for digests that have no, moderate or a full effect on transformation frequency;

FIG. 4 graphically represents the map of restriction enzyme cleavage sites in a random region of the B. subtilis genome for digests that have no, moderate or a full effect on transformation frequency;

FIG. 5 graphically represents the signatures of restriction enzyme digest transformation frequencies in a bar code format for the known location of the rifampicin resistance mutation in the rpoB gene and two random locations in the B. subtilis genome;

FIG. 6 graphically represents the map of restriction enzyme cleavage sites in the region of the known ciprofloxacin resistance mutation in the H. influenzae gyrA gene for digests that have no, moderate or a full effect on transformation frequency;

FIG. 7 graphically represents the map of restriction enzyme cleavage sites in a random region of the H. influenzae genome for digests that have no, moderate or a full effect on transformation frequency;

FIG. 8 graphically represents the signatures of restriction enzyme digest transformation frequencies in a bar code format at the known location of the ciprofloxacin resistance mutation in the gyrA gene and at two random locations in the H. influenzae genome;

FIG. 9 graphically represents the transformation frequency observed with various restriction enzyme digests of genomic DNA from a gyrA ciprofloxacin resistant H. influenzae mutant. Restriction enzyme names are indicated as labels above the bars whose heights represent the observed transformation frequencies. Classification of the digests into full- moderate- or no-effect categories is also indicated.

FIG. 10 graphically represents data obtained with a novobiocinresistant gyrB H. influenzae mutant. The results are presented in the same format as for FIG. 9;

FIG. 11 graphically represents data obtained with a spectinomycin-resistant rpS5 H. influenzae mutant. The results are presented in the same format as for FIG. 9;

FIG. 12 graphically represents data obtained with Abbott compound A-583-resistant fadL H. influenzae mutant. The results are presented in the same format as for FIG. 9;

FIG. 13 graphically represents data obtained with Abbott compound A-568-resistant acrB H. influenzae mutant. The results are presented in the same format as for FIG. 9;

FIG. 14 graphically represents the signatures of restriction enzyme digest transformation frequency in a bar code format for H. influenzae mutants resistant to ciprofloxacin, novobiocin, spectinomycin, and Abbott compounds A-583 and A-568 with resistance mutations in the gyrA, gyrB, rpS5, fadL, and acrB genes, respectively.

### DETAILED DESCRIPTION OF THE INVENTION

The present invention provides methods for identification of mutation locations in genomes by assessing the frequency with which linear DNA fragments, generated by restriction enzyme digestion of mutant genomic DNA, transform recipient cells and comparing the observed transformation frequencies for a set of restriction enzyme digests with the genome restriction map which is derived from the organism's complete or partial genome sequence information.

In one embodiment of the present invention a method is provided for identifying the location of a mutation in the genome of a particular organism, that method comprises the steps of: a) isolating DNA from an organism having a mutated phenotype, for example, drug resistance; b) treating the DNA with a panel of restriction enzymes to completely digest the DNA into fragments; c) introducing the fragments into a non-mutated host organism to transform the organism into a mutated organism that expresses the drug resistance phenotype; d) determining the transformation frequency by counting the number of the drug resistant organisms resulting in step (c), and (e) correlating the transformation frequency to the known locations of the restriction enzyme cleavage sites for the enzymes used in step (b), to provide information regarding the location of said mutation in the genome.

In general, transformation frequency decreases as fragment lengths and/or distances of mutations from fragment ends decrease. Thus, the smaller the fragment, or the closer a mutation is to the end of a fragment, the lower the transformation frequency. Thus, restriction enzyme digests that yield low transformation frequencies indicate close proximity of the mutation to such restriction sites. Correspondingly, restriction enzyme digests that exhibit high transformation frequencies indicate that the mutation is not close to sites for such enzymes. Examining the genome restriction map for regions that (1) contain clusters of cleavage sites for enzymes that decrease transformation frequencies, but (2) do not contain clusters of cleavage sites for enzymes that do not reduce transformation frequencies, provides a short list of candidate regions in the genome one of which most likely contains the mutation.

Transformation frequency as used herein, means, the number of colonies observed on Petri plates containing agar growth medium including a chemical component that inhibits the growth of non-resistant cells but does not inhibit growth of cells that are resistant to the chemical. A restriction site, as used herein, means a restriction enzyme cleavage site, and the terms can be used alternatively. Similarly, a restriction enzyme digest, as used herein, means the treatment of a genomic DNA sample with a restriction enzyme, resulting in particular genomic fragments defined by the identity of the restriction enzyme used in the digest. A restriction map, as used herein, means the series of locations of restriction endonuclease cleavage sites in a DNA sequence.

In some species of bacteria, in addition to the effects of the fragment length and distance to the end, there are also specific uptake signal sequences (USS) scattered throughout the genome that are required for efficient transformation. In these cases, restriction digests that result in the nearest USS sequences being cut off of the mutation-bearing fragment will have low frequency of transformation (an effect similar to the restriction site being close to the mutation). In these cases, the aforementioned examination of the genome would be for regions that (1) contain clusters of cleavage sites or cleavage sites that would result in a fragment devoid of a USS, for enzymes that decrease transformation frequencies, but (2) do not contain clusters of cleavage sites for enzymes that do not reduce transformation frequencies. This process yields a list of candidate regions in the genome, one of which most likely contains the mutation.

The dependence of transformation frequencies on fragment length, distance from fragment ends, and existence/effect of USS varies from organism to organism but, such relationships can be determined empirically by, for example, using mutant strains with mutations in known locations or appropriately constructed PCR products. This dependence has been assessed to varying extents in a few organisms. None of the reports suggest correlating transformation data with genomic restriction maps to identify locations of mutations (Belanger, A. E., et al., Antimicrob Agents Chemother Vol. 46, pages 2507-2512 (2002); Lataste, H., et al., Mol Gen Genet. Vol. 183, pages 199-201 (1981); Lee, M. S., et al., Appl Environ Microbiol. Vol. 65, pages 1883-1890 (1999); Lee, M. S., et al., Appl Environ Microbiol. Vol. 64, pages 4796-4802 (1998); Lau, P. C., et al., J Microbiol Methods. Vol. 49, pages 193-205 (2002); Zawadzki, P. and F. M. Cohan, Genetics Vol. 141, pages 1231-1243 (1995)).

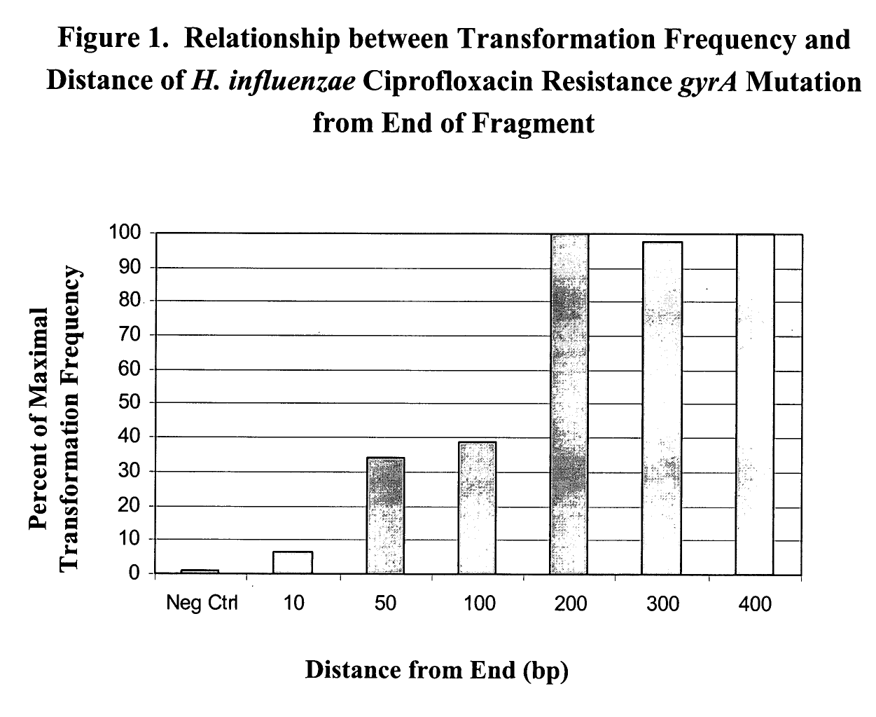

To assess the relationship between transformation frequency and distance of a mutation from the end of a fragment, PCR was used to generate fragments of constant length (1,000 bp) containing a ciprofloxacin resistance missense mutation in the H. influenzae gyrA gene at varying distances from the end of the fragment. Genomic DNA from a ciprofloxacin resistant strain was used as a positive control in the length dependence experiment, and DNA from a sensitive strain was used as a negative control. As the distance of the mutation from the end of the fragment decreased, the transformation frequency decreased, with significant decreases in transformation frequencies occurring between 100 and 200 bp and again between 10 and 50 bp from the end, as represented in FIG. 1.

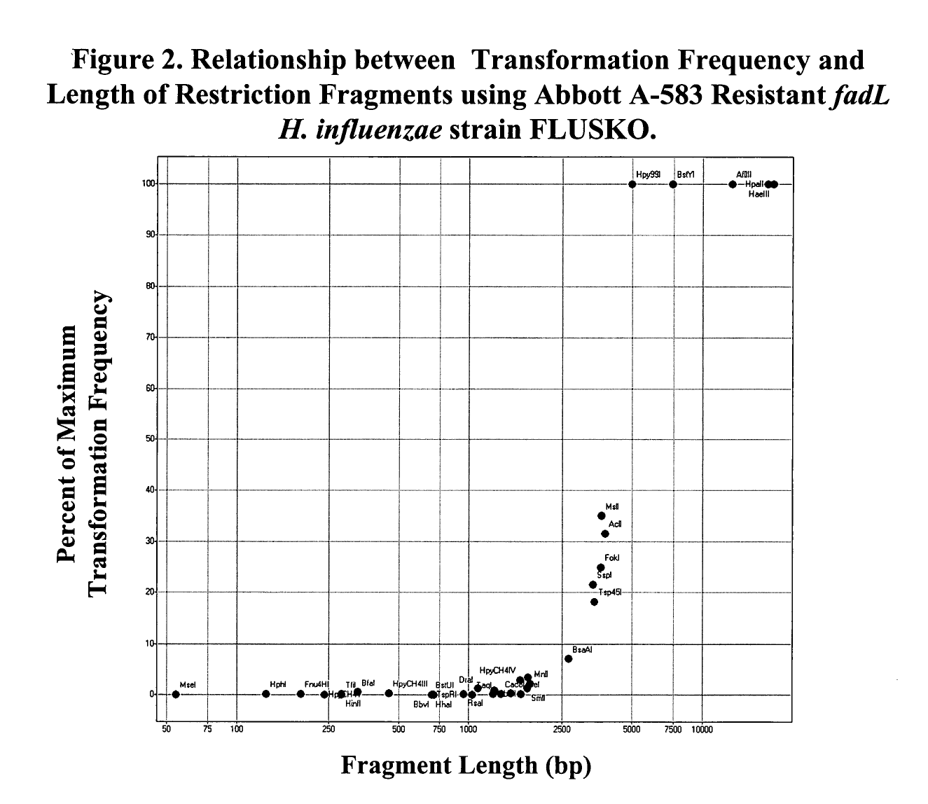

To assess dependence of fragment length on transformation frequency a control H. influenzae strain (FLUSKO) was constructed in which a resistance mutation was immediately adjacent to a USS uptake sequence. Thus, all restriction fragments would be able to gain entry into the cell via the USS sequence so decreases in transformation frequencies are not due to lack of USS-mediated DNA uptake. FIG. 2 shows the dependence of transformation frequency on DNA fragment length observed with restriction enzyme digests of DNA isolated from the FLUSKO control strain. As the fragment length approaches ˜2,500 bp the transformation frequency decreases dramatically. Below ˜1,500 bp the transformation frequency approaches zero.

The essential concept of the method is that for any given restriction enzyme digest, the size of the fragment containing the resistance mutation and the distance of the mutation to the end of the fragment are defined by the location of the surrounding restriction enzyme cleavage sites. The mapping procedure can be conceived of as a process of elimination in which digests that transform with high frequency indicate that the restriction enzymes cleavage sites are relatively far away from the mutation, while digests that transform with low frequency indicate that the restriction enzyme cleavage site are located close to the mutation. Regions of the genome that contain sites for high transformation frequency restriction enzymes are eliminated as potential locations of the mutation, while sites for restriction enzymes that give rise to low transformation frequencies are locations potentially near the resistance mutation. Each enzyme used in the method results in a reduction in the number of possible locations of the mutation thereby eliminating a substantial portion of the genome from consideration; although several enzymes are needed to completely narrow down to a single locus.

By blocking out sites for the subset of high transformation frequency enzymes and highlighting sites for the low transformation frequency enzymes, potential sites for the mutation can directly be identified on a printout of the genome restriction map.

In detail, the analysis is performed by first sorting the enzyme digest transformation data by the corresponding transformation frequencies. The enzymes are then classified into three categories: ‘Full Effect’, ‘Moderate Effect’ and ‘No Effect’ according to the extent of their effects on transformation frequency. On average, enzymes that decrease the transformation frequency to less than, or equal to, 0.3% of the maximal level are categorized as having a ‘Full Effect’ and thus likely cleave very close to the mutation. Enzymes that decrease the transformation frequency to between 0.4 and 1.3% of the maximum are binned into the ‘Moderate Effect’ category and thus likely cleave close to the mutation, but not as close as the ‘Full Effect’ enzymes. The remaining enzymes, which on average yield at least 2.1% of the maximal transformation frequency, are binned into the ‘No Effect’ category. Such enzymes likely do not cleave close to the mutation. Table 1 shows the average values and ranges for the three transformation effect categories in terms of the total numbers of transformants and the percent of maximal transformation frequency. These are empirical values obtained from analysis of five H. influenzae resistance mutations; ciprofloxacin resistance in gyrA, novobiocin resistance in gyrB, spectinomycin resistance in rpS5, A-583 resistance in fadL, and A-568 resistance in acrB. Representative data for these experiments is provided in Example 3.

TABLE 1

| | Resistance | Values used to assign enzyme transformation effects | | | | | |
|---|---|---|---|---|---|---|---|
| | mutation | Percent of maximum transformation efficiency | | | Number of transformants* | | |
| Compound | gene | Full effect | Middle effect | No effect | Full effect | Middle effect | No effect |
| Ciprofloxacin | gyrA | 0.0-0.2 | 0.3-1.3 | ≧2.8 | ≦1,300 | 2,800-10,700 | ≧22,700 |
| Novobiocin | gyrB | 0.0-0.4 | 0.7-2.0 | ≧4.2 | ≦1,600 | 5,900-32,000 | ≧67,000 |
| Spectinomycin | rpS5 | 0.0 | 0.1-0.6 | ≧0.7 | ≦720 | 1,100-8,500 | ≧10,000 |
| Compound-583.1 | fadL | 0.0 | 0.1-0.7 | ≧0.7 | ≦400 | 500-6,200 | ≧14,000 |
| Compound-568.1 | acrB | ≦0.9 | 1.0-1.9 | ≧15.3 | ≦15,160 | 16,160-32,160 | ≧259,160 |
| | Averages: | ≦0.3 | 0.4-1.3 | ≧2.1 | ≦3800 | 5,300-18,000 | ≧75,000 |
| | Extremes: | 0-≦0.9 | 0.1-1.9 | ≧0.7-≧15.3 | ≦400-≦15,200 | 500-32,200 | ≧10,000-≧259,160 |

In another embodiment of the present invention, a computer analysis program is used to compare the observed transformation frequency data with a genome restriction enzyme cleavage map to identify the location of the mutation. This allows for a rapid identification of the genome locations that best fit the transformation data, meaning that region of the genome where the location of the restriction enzyme cleavage sites is most highly correlated with the transformation data given the dependence of the transformation frequency on species-specific characteristics of restriction fragment length, mutation position and other possible sequence characteristics. Enzymes of the three classes, categorized using the experimental data, are entered into a computer program that scans the H. influenzae genome sequence (GenBank Accession number L42023) in steps of 10 bp. User defined variables are also entered including fragment length and mutation distances from fragment end parameters that are determined from control experiments to set cutoff values for enzymes predicted to have full-, moderate-, or no effect on transformation frequencies. Two of these parameters are the sizes of windows surrounding the 10 bp test location. One is a small window within which mutations would be too close to a fragment end to yield significant numbers of transformants. Surrounding this, a larger window is set within which the mutation would be far enough away from the fragment end to allow transformation, but still too close for high frequency transformation. Enzymes that cleave within the small window would be predicted to have a ‘full effect’ on the transformation frequency, dropping it to nearly zero. Enzymes that cleave between the small and large window would be expected to give rise to low but detectable numbers of transformants and thus have a moderate effect on transformation frequency. The algorithm also takes into account the dependence of transformation frequency on fragment length. Two additional parameters define the length of fragments that either do not effect transformation, or have a full to moderate effect on transformation frequency. The algorithm also can take into account the presence or absence of USS DNA uptake sequences on the fragment.

At each 10 bp step the program scans the region between the test location and the small window to identify sites for restriction enzymes that would be expected to dramatically decrease the transformation frequency and thus be sites for ‘full effect’ enzymes. Next, it scans the sequence between the small and large window to identify sites for restriction enzymes that would be expected to decrease the transformation frequency to a lesser extent and thus be sites for ‘moderate effect’ enzymes. The program then scans the surrounding region for the location of enzyme cleavage sites within the boundaries set by the values entered for the fragment length dependence variables. It calculates the length of the fragment surrounding the test location and compares the length to the variable values. Fragments longer than the cutoff for no effect enzymes are identified. Enzymes that give rise to such fragments would not significantly decrease the transformation frequency. Similarly, enzymes that would give rise smaller fragments—and are expected to give rise to few or no transformants—are also identified. The user can also request the program to determine whether or not USS sequences are present on the fragments. The absence of USS sequences dramatically decreases the transformation frequency so enzymes that yield such fragments are classified as ‘full-effect’ enzymes. The program then compares these lists of enzymes with predicted transformation effects to the observed enzyme transformation effects and calculates the number of correct enzyme matches and incorrect mismatches for the test location.

The preferred values for use in H. influenzae are determined from the control experiments described above (FIG. 1 and FIG. 2). The small window is set at 100 bp centered around the 10 bp test location encompassing 50 bp on each side. Mutations that are within 50 bp of the end of a fragment essentially do not yield transformants (FIG. 1). Sites for restriction enzymes within 50 bp of the test location are expected to have a ‘full-effect’ on transformation, i.e., very few or no transformants obtained. A larger window of 300 bp, 150 bp on either side, is also set to identify sites for putative ‘moderate-effect’ enzymes which give rise to decreased numbers of transformants, but significantly higher than background. The preferred values for the length dependence parameters are 1,500 bp for full to moderate effect enzymes and 2,500 bp for no effect enzymes.

After storing the numbers of correct and incorrect matches at a particular site the program then moves down the sequence 10 bp and repeats the analysis. The program advances along the entire genome and generates a list that can be sorted to identify the locations that contain the most correct and fewest incorrect enzyme matches with the empirical data. The output can be limited by visualizing only those locations that match the empirical data by some percentage, 80% being the preferred cutoff.

Sometimes the region containing a mutation may not be the absolute best match to the empirical data. A very small number of enzymes could provide unexpected results due to rare differences between the reference genomic DNA sequence and the actual sequence in the bacterial strain being used, so that a particular restriction site near the mutation may be either created, or obliterated. Other times the restriction digest may not work with perfect fidelity, leaving the particular site near the mutation uncut, or accidentally cut where it should not. These are very rare occurrences so that, as shown in Table 2, the location of the actual mutation is typically the highest ranked location in the entire genome, and should nearly always be at least in the top 10. Given the ease of PCR technology it is simple to merely follow the method of the invention with a screen of the top 10 locations by testing the ability of PCR products for each of the 10 locations by transformation to identify the one that confers resistance with high frequency. This serves to identify the correct location of the mutation. Using this same PCR product the precise location and identity of the mutation can be determined by DNA sequencing.

It should be noted that, although the procedure of the instant invention does not directly identify the exact location and identity of the mutated nucleotide, in a preferred embodiment of the present application, the procedure could be coupled with other methods to identify the precise location and identity of the mutation within the potential locations, which is typically only 100 to 300 base-pairs long (Table 2; see Example 3 for additional details). Table 2 indicates the location of mutations relative to the numbering of the reference genome of H. influenzae Rd (GenBank accession number L42023; Fleischmann, R. D. et al., Science Vol. 269(5223), pages 496 to 512 (1995)). To identify the exact location of the mutation, the previously identified locations can be amplified by PCR using genomic DNA from the mutant as a template, and tested for the ability to transform and confer the mutant phenotype on non-mutant host cells. The PCR product that confers the mutant phenotype with high frequency was amplified from the region of the template genomic DNA that contains the mutation. The exact location of the mutated nucleotide can then be determined by sequencing of the PCR product, and comparing the sequence with the sequence in the genome database or with the sequence of the analogous PCR product generated from non-resistant non-mutant genomic DNA template.

TABLE 2

| | | | Rank of | Region of | | | Distance of | | Distance of | |
|---|---|---|---|---|---|---|---|---|---|---|
| | Resistance | Location of | mutation | genome identi- | Length | Mutation | mutation from | | mutation to | USS On/ |
| | mutation | mutation in | location in | fied by | of | in | end of region | | closest | Off in |
| Compound | gene | genome | output list | analysis | Region | region | Left | Right | USS,bp | analysis |
| Ciprofloxacin | gyrA | 1,344,100 | 1st | 1,343,859-1,344,160 | 301 | Yes | 241 | 60 | 75 | On |
| Novobiocin | gyrB | 587,579 | 6th | 587,520-587,760 | 240 | Yes | 59 | 181 | 615 | On |
| Spectinomycin | rpS5 | 847,961 | 1st | 847,930-848,010 | 80 | Yes | 31 | 49 | 3,163 | Off |
| Compound-583.1 | fadL | 422,238 | 1st | 422,260-422,340 | 80 | No | 22 | 102 | 365 | On |
| Compound-568.1 | acrB | 950,222 | 1st | 949,980-950,270 | 290 | Yes | 242 | 48 | 1,413 | On |

The method relies on DNA purification, restriction enzyme digestion and transformation techniques that are well known in the art. DNA purification and restriction enzyme digestion methods are well established (Molecular Cloning: A Laboratory Manual, 2001, Third Edition, Sambrook, J. and Russell, D., Cold Spring Harbor Laboratory Press, Cold Spring Harbor, N.Y.). Transformation methods are available for many organisms and are continually being developed (Lorenz, M. G., and Wackemagel, W. Microbiol Rev. Vol. 58, pages 563-602 (1994); BTX Instrument Division, Harvard Apparatus, Inc., Holliston, Mass.; Bio-Rad Laboratories, Hercules, Calif.).

For the method of the present invention, genomic DNA samples are treated with restriction enzymes to digest the DNA into fragments with lengths defined by the location of the restriction enzyme cleavage site in the genome sequence. Equal amounts of digested DNA are then used to transform a non-mutated non-resistant host strain. The transformation mixture is plated on agar containing the antibacterial agent to select for resistant colonies that have acquired the mutation by transformation. The number of resistant colonies (transformants) is affected by several factors, the most critical of which, for the purpose of this method, are (1) the distance of the mutation from the end of the DNA fragment, (2) the length of the DNA fragment, and in some species, (3) the presence or absence of signal sequences required for DNA uptake (USS, uptake signal sequences; (e.g., H. influenzae, H. parinfluenzae and N. gonorrhoeae; Smith, H. O., et al., Res Microbiol. Vol. 150, pages 603-616, (1999)). Fragments that do not contain USS sequences transform with extremely low frequencies (essentially independent of fragment length). Fragments that contain USS sequences transform with frequencies dependent on the size and distance of the mutation from the fragment end. The dependence of transformation frequency on fragment size, and mutation distance from fragment ends, varies from organism to organism but can be established empirically by assessing transformation frequencies with control strains containing mutations in known locations, or by using PCR products of varying lengths containing a mutation in the middle of the fragment. Similarly, data from control mutations and/or a set of PCR products of constant length with the mutation at different positions from the end of the fragment are used to assess the dependence of transformation frequency on the distance of the mutation from the end of a fragment.

In another embodiment of the present invention, the method is able to identify the location of mutations that confer phenotypes other than resistance to antibacterial compounds. Such mutations include, but are not limited to, those that improve the production of human or animal biologicals such as insulin, growth hormone and antibodies, as well as industrial enzymes used in the production of cheese, the clarification of apple juice, laundry detergents, pulp and paper production and the treatment of sewage. Also included are mutations that enhance the production of secondary metabolites with pharmacological activities such as antibiotics, and other metabolites useful in the treatment of hypertension, obesity, coronary heart disease, cancer and inflammation. Additional secondary metabolites of industrial importance include organic acids and chemicals such as citric, malic and ascorbic acids, and acetone, methanol, butanol, ethanol and detergents. Also included are mutations that enhance the production of amino acids such as monosodium glutamate, and also carbohydrates. Additional mutations include those that enhance a microbial strain's ability to degrade and detoxify hydrocarbons and halogenated hydrocarbons. Further additional mutations include those that improve the activity of microbial strains used in assays to detect microbial contaminants in food, evaluation of natural or synthetic agents for the prevention of disease, deterioration or spoilage, determination of minute quantities of vitamins or amino acids in food samples, development of preservatives for control of food spoilage, and development of procedures for control of deterioration in cosmetics, steel, rubber, textiles, paint and petroleum products (Society for Industrial Microbiology, www.simhq.org).

In an additional embodiment of the present invention, the method is used with one or more organisms for which transformation methods are available. These include bacteria, yeast, fungi, Plasmodia, and multicellular organisms, preferably mammalian.

Bacteria most suitable for the method include those that are transformable (naturally, by electroporation or treatment with salts) and for which the entire sequence of their genomic DNA has been determined. Numerous bacterial species can be made to take up exogenous DNA and incorporate the DNA into their genome/chromosome by homologous recombination. Certain bacteria are known to naturally take up DNA from the environment. More than 40 naturally transformable bacterial species have been identified, including Hemophilus influenzae, Hemophilus parinfluenzae Streptococcus pneumoniae, Streptococcus mutans, Streptococcus sanguis, Bacillus subtilis, Nisseria gonorrhoeae, Nisseria meningitidis, Helicobacter pylori, Pseudomonas stutzeri, Campylobacter species and Synechocystis species (Lorenz, M. G., and Wackernagel, W., Microbiol Rev. Vol. 58, pages 563-602 (1994)). Other bacteria can be made to take up DNA by electroporation (BTX Instrument Division, Harvard Apparatus, Inc., Holliston, Mass.; Bio-Rad Laboratories, Hercules, Calif.) or by exposure to certain salts (Molecular Cloning: A Laboratory Manual, 2001, Third Edition, Sambrook, J. and Russell, D., Cold Spring Harbor Laboratory Press, Cold Spring Harbor, N.Y.). Genome sequences of bacterial species continue to be determined and methods for transforming bacteria also continue to be developed. The current list of bacterial species with complete genome sequence information and transformation protocols include the following: Agrobacterium tumefaciens, Caulobacter crescentus, Listeria monocytogenes, Borrelia burgdorferi, Brucella melitensis, Campylobacter jejuni, Clostridium perfringens, Corynebacterium glutamicum, Escherichia coil, Enterococcus faecalis, Helicobacter pylori, Mycoplasma pneumoniae, Mycoplasma genetalium, Pasteurella multocida, Pseudomonas aeruginosa, Pseudomonas putida, Pseudomonas syringae, Rickettsia prowazekii, Salmonella enterica, Salmonella typhimurium, Staphylococcus aureus, Streptococcus Pneumoniae, Streptococcus pyogenes, Xanthomonas campestris pv. canpestris, Yersinia pestis, Bacillus subtilis, Deinococcus radiodurans, Haemophilus influenzae, Lactococcus lactis, Neisseria meningitidis, Nostoc s.p, Streptococcus mutans, Streptomyces coelicolor, and Synechocystis sp.

Another embodiment of the method is identification of the location of mutations that confer phenotypes in organisms that can be transformed with linear DNA fragments by homologous recombination but for which a partially complete genome map of restriction enzyme cleavage sites is available from the partially complete genome sequence information. Therefore, the availability of the entire genome sequence is not absolutely necessary for the method. In such cases, candidate mutation locations that best fit the available sequence data can be identified and subsequently tested, although the likelihood of successfully identifying the location of the mutation is lower than for completely sequenced genomes. Many transformable bacteria have partial genome DNA sequence data deposited in databases such as GenBank. The following bacterial species are transformable by electroporation but their complete genome sequences currently are not available: Acetobacter xylinum, Acholeplasma laidlawii, Acinetobacter baumannii, Actinobacillus pleuropneumoniae, Actinomyces vvscosus, Agrobacterium rhizogenes, Amycolatopsis mediterranei, Amycolatopsis orientalis, Anabaena sp, Azospirillum brasilense, Azotobacter vinelandii, Bacillus cereus, Bacillus parapertussis, Bacillus thuringiensis, Bacillus licheniformis, Bacillus sphaericus, Bacillus thuringiensis, Bacteroides fragilis, Bordetella pertussis, Bradyhizobium japonicum, Brevibacterium flavum, Brevibacterium lactofermentum, Brucella abortus, Butyrivibrio fbrisolvens, Citrobacter freundii, Clavibacter michiganensis, Clostridium botulinum, Clostridium cellulolyticum, Clostridium difficile, Cyanobacterium chroococcidiopsis, Cytophaga johnsonae, Dichelobacter nodosus, Enterobacter aerogenes, Enterobacter agglomerans, Enterococcus hirae, Erwinia carotovora, Francisella sp, Fremyella diplosiphon, Giardia lambia, Klebsiella pneumoniae, Lactobacillus acidophilus, Lactobacillus casei, Lactobacillus delbrueckii, Lactobacillus fermentum, Lactobacillus gasseri, Lactobacillus helveticus, Lactobacillus plantarum, Lactobacillus salivarius, Lactobacillus teuteri, Legionella pneumophila, Leptospira biflexa, Leuconostoc sp, Methylobacterium extorquens, Mannheimia haemolytica, Methylophillus spp, Mycobacterium aurum, Mycobacterium bovis, Mycobacterium smegmatis, Myxococcus xanthus, Pasteurelia haemolytica, Pasteurella trehalosi, Pediococcus acidilactici, Propionibacterium jensenii, Proteus sp, Pseudomonas oleovorans, Rhizobium leguminosarum, Rhodococcus equi, Rhodopseudomonas viridis, Rhodospirillum molischianum, Rochalimaea quintana, Rubrivivax gelatinosus, Saccharopolyspora erythraea, Salmonella senftenburg, Seratia sp, Serpula hyodysenteriae, Spirulina platensis, Streptococcus cremoris, Streptococcus parasanguis, Streptococcus salivarus, Streptococcus sanguis, Sulfolubus Shibatae, Synechococcus sp., Toxoplasma gondii, Vibrio anguillarum, Vibrio sp, Yersinia pseudotuberculosis, Yersinia enterocolitica and, Zymomonas mobilis.

Another embodiment of the method is identification of the location of mutations that confer phenotypes in yeast and fungi that can be transformed by electroporation, protoplasting or exposure to salts (Zymo Research, Orange, Calif.; BTX Instrument Division, Harvard Apparatus, Inc., Holliston, Mass.; Bio-Rad Laboratories, Hercules, Calif.; Gietz, R. D. and R. A. Woods., Methods in Enzymology Vol. 350, pages 87-96 (2002); Moreno S, et al., Methods Enzymol. Vol. 194, pages 795-823 (1991); Alfa, C., et al., (1993) Experiments with fission yeast. Cold Spring Harbor Laboratory Press, Cold Spring Harbor, N.Y.). Transformable yeast and fungi for which complete genome sequence information is currently available include Aspergillus fumigatus, Asperfillus nidulans, Aspergillus parasiticus, Aspergillus terreus, Cryptococcus neoformans, Neurospora crassa, Saccharomyces cerevisiae, Schizosaccharomyces pombe, and Candida albicans. Thus, the method of this application may be applicable to these organisms. Although their genome sequences are not currently complete, transformation protocols have also been developed for Candida utilis, Candida glabrata, and Candida oleophila (Rodriguez, L., et al., FEMS Microbiol Lett., Vol. 165(2), pages 335-340 (1998); Cormack, B. P. and Falkow, S., Genetics, Vol 151(3), pages 979-987 (1999); Yehuda, H., et al., Curr Genet. Vol. 40(4). pages 282-287 (2001)). Thus, the method of this application may be applicable to these organisms with incomplete genome sequence information that is available in DNA sequence databases.

Another embodiment of the method is identification of the location of mutations that confer phenotypes in additional unicellular eukaryotic organisms such as the malaria parasite Plasmodium falciparum for which complete genome sequences and homologous recombination transformation methods are available (Menard, R. and Janse, C. Methods, Vol Oct. 13(2). pages 148-157 (1997)).

Another embodiment of the method is identification of the location of mutations that confer phenotypes in multicellular organisms for which complete genome sequences and homologous recombination transformation methods are available. This is currently the case for the fruit fly Drosophila melanogaster (Rong, Y. S., et al., Genes Dev., Vol. 16(12), pages 1568-1581 (2002); Rong, Y. S., and Golic, K. G. Genetics., Vol. 157(3), pages 1307-1312 (2001); Rong, Y. S. and Golic, K. G. Science, Vol. 288(5473), pages 2013-2018 (2000)). Additional multicellular organisms with completed genomes but for which transformation procedures with useful frequencies of homologous recombination are not yet available include the mosquito Anopheles gambiae, the plant Arabidopsis thaliana, the nematode worm Caenorhabditis elegans, and the parasite Encephalitozoon cuniculi. When protocols are developed that enable transformation via homologous recombination with sufficient frequency, another embodiment is to identify mutations that confer phenotypes in these organisms.

Another embodiment of the method is identification of the location of mutations that confer phenotypes in human and mouse cells. Given that the human and mouse genome sequences are nearly complete, and protocols exist for homologous recombination transformation of embryonic stem cells, the method could also be applied to identify mutations that confer phenotypes in mouse and possibly human embryonic stem cells (Templeton, N. S. et al., Gene Ther. Vol. 4(7), pages 700-709 (1997), Zwaka, T. P. and Thomson, J. A., Nat Biotechnol. Vol. 21(3), pages 319-321 (2003); Capecchi, M. R. Sci Am. Vol. 270(3), pages 52-59 (1994); Capecchi, M. R. Science, Vol. 16;244(4910), pages 1288-1292 (1989); Capecchi, M. R. Trends Genet. Vol. 5(3), pages 70-76 (1989)).

The preferred embodiment of the present method is for identification of the location of drug resistance mutations in bacterial species that (1) can be transformed with linear DNA fragments by homologous recombination selecting drug resistant transformants, and (2) for which the complete genome map of restriction enzyme cleavage sites is available from the complete genome sequence information. The organisms of the preferred embodiment are Hemophilus influenzae, Bacillus subtilis and, Streptococcus pneumoniae for which natural transformation methods and complete genome sequence data are available. Any one of the available restriction enzymes can be suitable for the method.

The preferred set of restriction enzymes for these organisms are subsets of the following: AciI, AclI, AfIII, AluI, ApoI, AseI, BbvI, BfaI, BsaAI, BsaHI, BsaJI, BsrFI, BssKI, BstUI, BstYI, Cac8I, DdeI, FnuHI, FokI, HaeIII, HhaI, HinfI, HpaII, HphI, Hpy188I, Hpy99I, HpyCH4III, HpyCH4IV, HpyCH4V, MaeIII, MboII, MnlI, MseI, MslI, NlaIII, NlaIV, RsaI, Sau3AI, Sau96I, SfaNI, SfcI, SmlI, SspI, TaqI, TfiI, TseI, Tsp45I, Tsp509I, and TspRI. The frequency of enzyme cleavage sites in genomic DNA from each organism is used as a guiding factor in deciding which enzymes to use in the method. The dependence of transformation frequency on DNA fragment length and distance of a mutation from a fragment end for the preferred organisms serves as a guide for selecting enzymes (Table 3). The preferred enzymes were selected since, for the preferred organisms, they cut the genomic DNA into fragments that range in size from 300 to 2000 base pairs. Enzymes that cut infrequently (>2000 base pair average distance) generally cut far enough away from most mutation sites that they will rarely affect transformation frequencies, while enzymes that cut too frequently (<300 base pair average distance) will almost always interrupt transformation frequencies for any particular mutation. For example, the enzyme BsrFI cuts about every 9525 bases in H. influenzae, but in B. subtilis it cuts about every 1044 bases. This enzyme would not be an ideal one to use for H. influenzae, but it would be optimal for B. subtilis. The preferred set of enzymes yield digests that transform with a wide variety of transformation frequencies. It can, however, be useful to have a few infrequent cutting enzymes in the analysis, since an affect by one of them can be very advantageous in separating the true mutation site from the background loci.

TABLE 3

Preferred list of restriction enzymes
used in the examples

| | | Average Fragment Length | | |
|---|---|---|---|---|
| Enzyme | Recognition Site | H. influenzae | S. pneumoniae | B. subtilis |

| | | | | |
|---|---|---|---|---|
| AciI | CCGC | 355 | 918 | 250 |
| AdII | AACGTT | 2163 | 4153 | 3973 |
| AfIIII | ACPuPyGT | 2259 | 2665 | 2104 |
| AluI | AGCT | 324 | 204 | 189 |
| ApoI | PuAATTPy | 284 | 427 | 559 |
| AseI | ATTAAT | 938 | 3441 | 2584 |
| BbvI | CCATC | 1251 | 1406 | 639 |
| BfaI | CTAG | 937 | 341 | 1355 |
| BsaAI | PyACGTPu | 1515 | 2918 | 2614 |
| BsaHI | GPuCGPyC | 7457 | 4690 | 1592 |
| BsaJI | CCNNGG | 1316 | 609 | 678 |
| BsrFI | PuCCGGPy | 9525 | 7028 | 1044 |
| BssKI | CCNGG | 1910 | 1155 | 522 |
| BstUI | CGCG | 661 | 1342 | 497 |
| BstYI | PuGATCPy | 2158 | 2275 | 1643 |
| Cac8I | GCNNGC | 416 | 554 | 267 |
| DdeI | CTNAG | 608 | 336 | 460 |
| FnuHI | GCNGC | 378 | 540 | 179 |
| FokI | GGATG | 1924 | 1121 | 912 |
| HaeIII | GGCC | 1810 | 885 | 444 |
| HhaI | GCGC | 562 | 891 | 342 |
| HinfI | GANTC | 583 | 309 | 313 |
| HpaII | CCGG | 3097 | 2476 | 290 |
| HphI | GGTGA | 1770 | 1204 | 1093 |
| Hpy188I | TCNGA | 480 | 315 | 245 |
| Hpy99I | CG(AT)CG | 1578 | 1683 | 1324 |
| HpyCH4III | ACNGT | 421 | 325 | 351 |
| HpyCH4IV | ACGT | 323 | 510 | 465 |
| HpyCH4V | TGCA | 182 | 297 | 252 |
| MaeIII | GTNAC | 454 | 373 | 455 |
| MboII | GAAGA | 720 | 471 | 530 |
| MnII | CCTC | 828 | 402 | 381 |
| MseI | TTAA | 107 | 192 | 183 |
| MsII | CAPyNNNNPuTG | 1291 | 1530 | 1143 |
| NlaIII | CATG | 881 | 313 | 256 |
| NlaIV | GGNNCC | 1808 | 790 | 661 |
| RsaI | GTAC | 514 | 543 | 540 |
| Sau3AI | GATC | 375 | 571 | 234 |
| Sau96I | GGNCC | 2307 | 1090 | 760 |
| SfaNI | GCATC | 1103 | 1314 | 902 |
| SfcI | CTPuPyAG | 2200 | 1386 | 1679 |
| SmII | CTPyPuAG | 1670 | 1115 | 1612 |
| SspI | AATATT | 899 | 1597 | 1875 |
| TaqI | TCGA | 514 | 422 | 385 |
| TfiI | GA(AT)TC | 715 | 484 | 403 |
| TseI | GC(AT)GC | 635 | 700 | 320 |
| Tsp45I | GT(CG)AC | 1236 | 757 | 782 |
| Tsp509I | AATT | 79 | 130 | 168 |
| TspRI | nnCA(CG)TGnn | 860 | 967 | 731 |

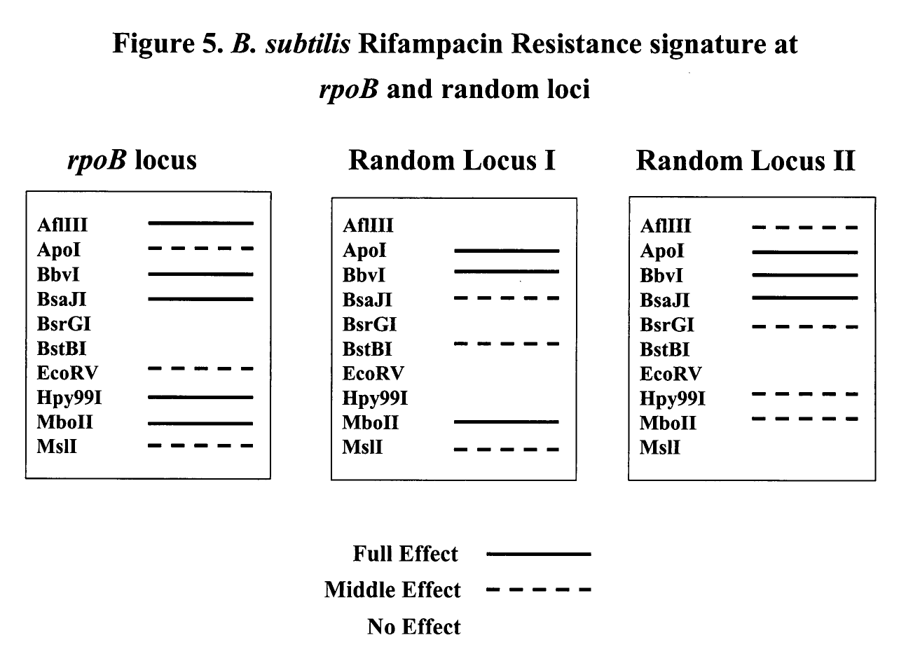

As discussed, following the acquisition of transformation frequency data, which is categorized as (1) full effect enzymes which maximally reduce the transformation frequency, (2) no effect enzymes which do not significantly affect the transformation frequency and, and (3) moderate effect enzymes which show an intermediate effect on transformation frequencies, the genome restriction map is analyzed to find the location that best fits the transformation data. Positions in the genome are evaluated as a potential location for the mutation, the local restriction map around each nucleotide is scanned to identify which bin the enzymes of the test set would fall into, with full effect enzymes being closest to the nucleotide, moderate effect enzymes being further away and no effect enzymes being the farthest away from the candidate nucleotide. In this way a signature is developed for the local restriction map encompassing the candidate nucleotide. This signature can be envisioned as a bar code. The bar code for each candidate nucleotide position is then compared to the experimentally obtained bar code. The bar codes most similar to the experimental bar code correspond to potential locations for the mutation (FIGS. 5, 8, and 14).

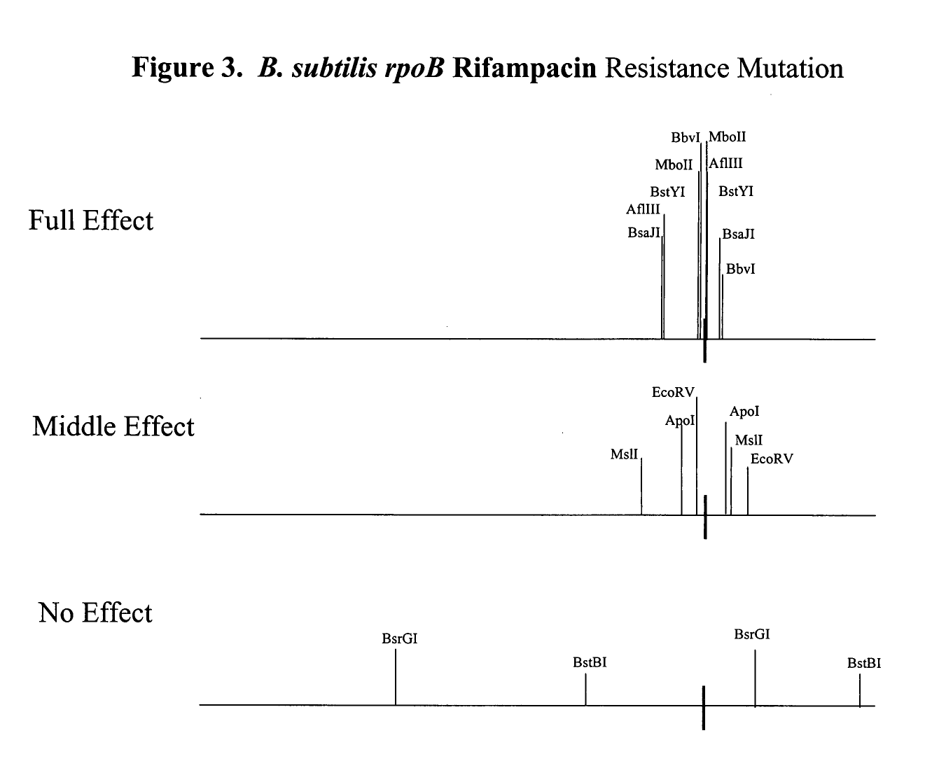

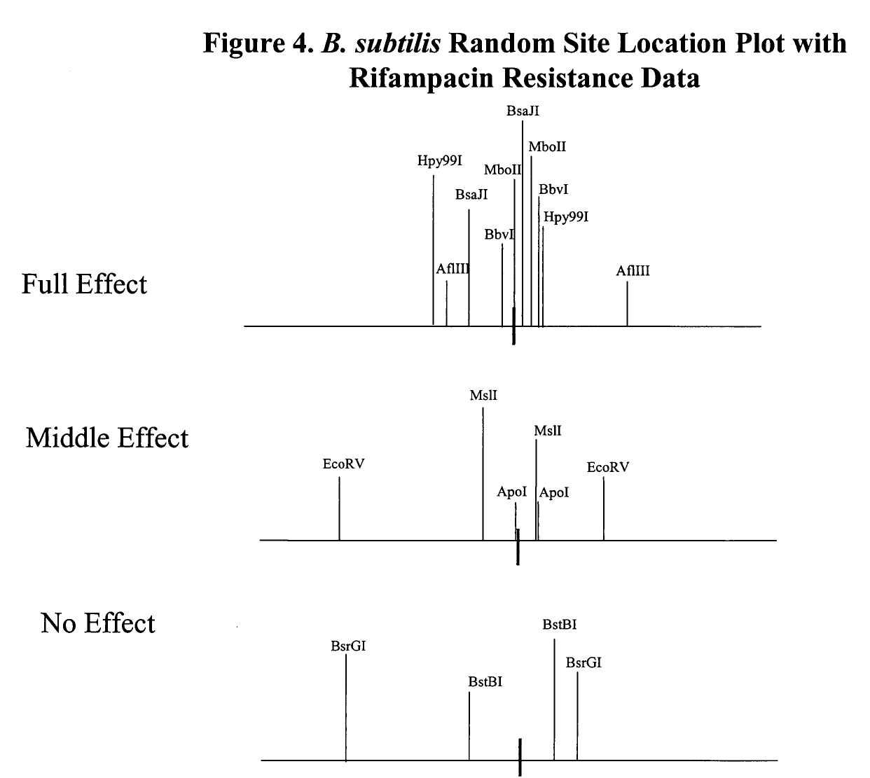

By way of example, representative data for mapping a rifampicin resistance mutation in B. subtilis by measuring differences in transformation frequencies with various enzyme digests is shown in Table 4. A detailed description of how this data was generated can be found hereinafter in Example 1. The restriction map of the region surrounding the B. subtilis rifampicin resistance mutation in the rpoB gene is shown in FIG. 3. For comparison, restriction sites surrounding a random region in the B. subtilis genome are shown in FIG. 4. Note how sites for the experimentally observed full effect enzymes cluster around the location of the mutation in the rpoB gene represented by the heavy vertical line, while sites for the moderate effect enzymes are less concentrated around the line and sites for no effect enzymes are generally far from the line. In contrast, for the random region, the sites for the full, moderate and no effect enzymes do not exhibit the correspondence between transformation frequency and proximity to the mutation found in the rpoB gene. The corresponding bar code representation of the data is shown in FIG. 5. Note how the bar codes for the random loci are distinct from the correct bar code in the rpoB gene.

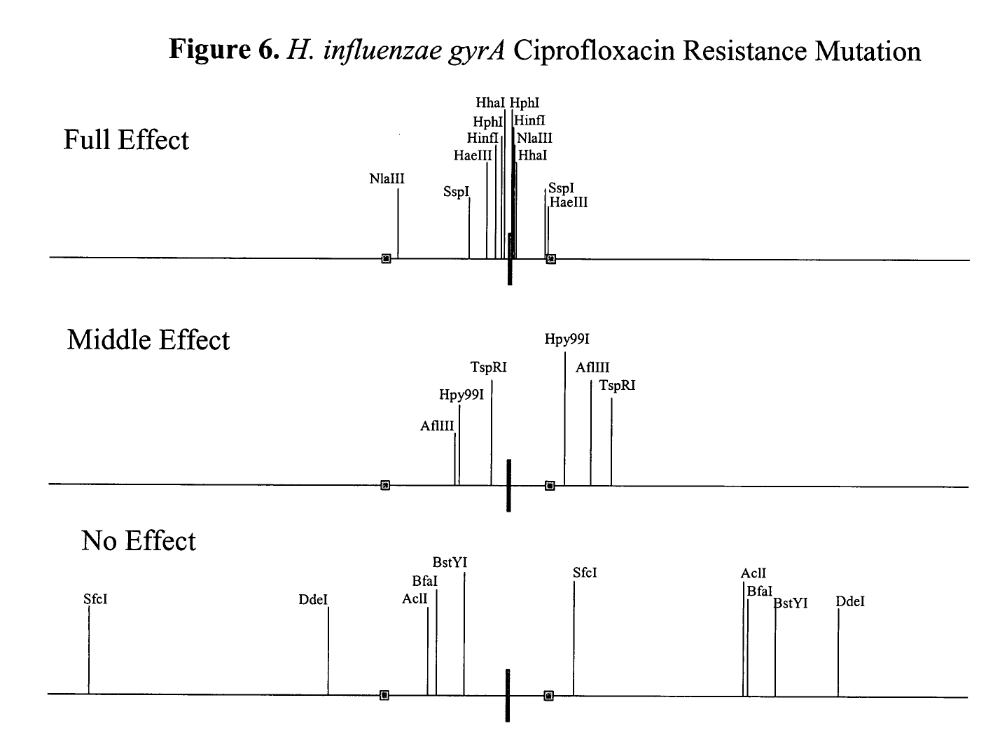

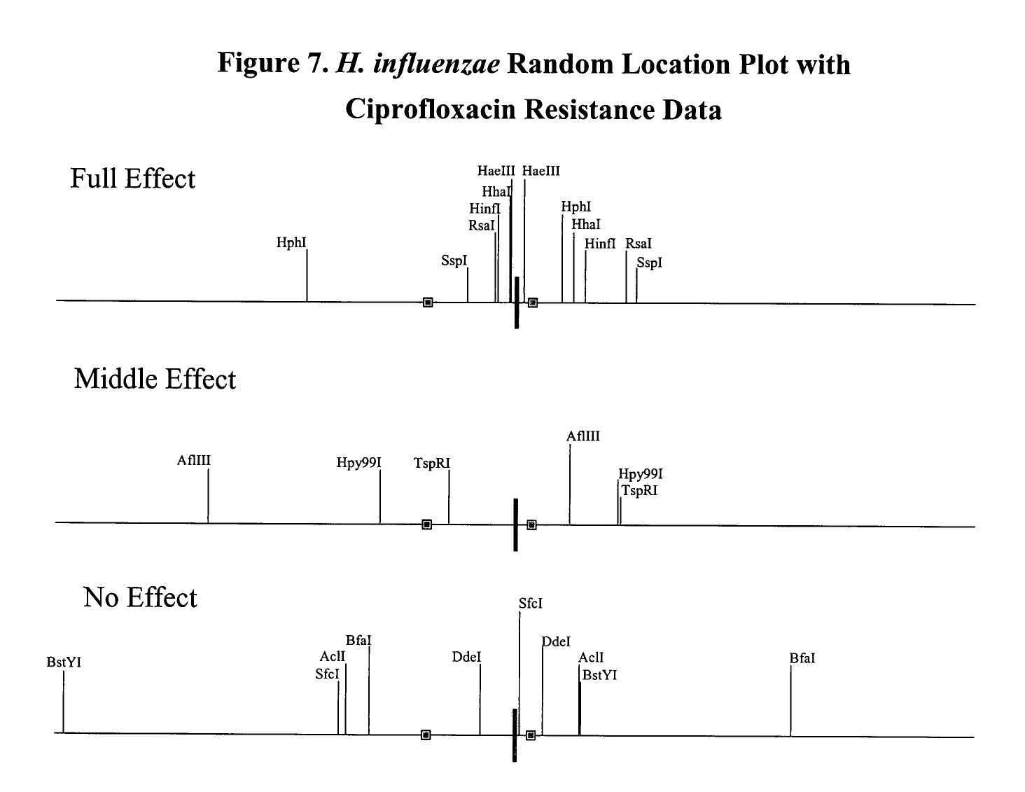

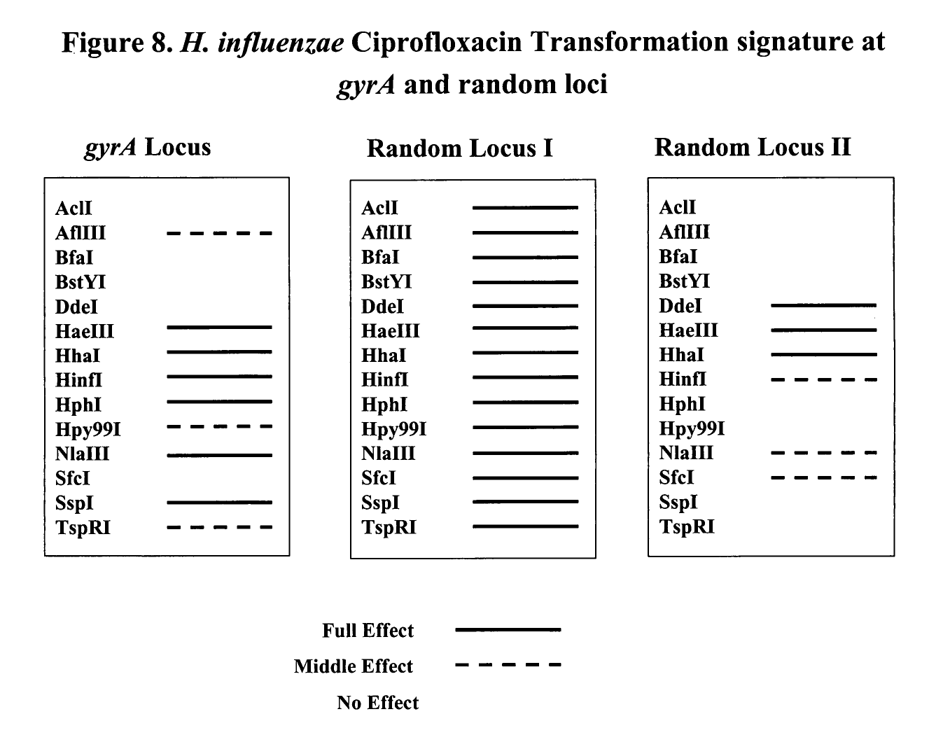

Also by way of example, representative data for mapping a ciprofloxacin resistance mutation in H. influenzae by measuring differences in transformation frequencies with various enzyme digests is shown in Table 5. A detailed description of how this data was generated can be found hereinafter in Example 2. This example is slightly more complicated due to the requirement that DNA is only taken up by H. influenzae if it contains uptake signal sequences (USS). The restriction map of the region surrounding the H. influenzae ciprofloxacin resistance mutation in the gyrA gene is shown in FIG. 6. For comparison, restriction sites in the region surrounding a random region in the H. influenzae genome are also shown in FIG. 7. The small gray boxes indicate the location of uptake signal sequences and the heavy vertical line indicates the location of the ciprofloxacin resistance mutation in gyrA, or a candidate location in a random region of the genome. As observed with the B. subtilis rifampicin mutation, restriction sites for experimentally observed full effect enzymes cluster around the heavy vertical line representing the location of the mutation. Note that all the full effect enzyme sites cluster between the uptake signal sequences, thus these fragments do not contain uptake signal sequences and so are not taken up by the cells thus transformants are not observed. The moderate and no effect enzyme sites flank both the mutation and at least one uptake signal sequence so they are taken up by cells and yield transformants. The moderate effect enzymes are shorter and thus transform with lower frequency than the longer fragments generated with no effect enzymes. The corresponding bar code representation of the data is shown in FIG. 8. Note how the bar codes for the random loci are distinct from the correct bar code in the gyrA gene.

### EXAMPLES

The present invention will be further clarified by the following examples, which are only intended to illustrate the present invention and are not intended to limit the scope of the present invention.

### Example 1

### Determination of the Location of a Rifampicin Resistance Mutation in B. subtilis

DNA isolation, Restriction Enzyme Digestion, and Transformation

Chromosomal DNA was isolated from B. subtilis rifampicinresistant strain R5 that has a mutation in the rpoB gene that confers resistance to rifampicin. Samples of the purified DNA were completely digested with an appropriate amount of restriction enzyme to yield completely digested DNA using the buffer and temperature recommended by the manufacturer. Portions of the DNA digests were analyzed by agarose gel electrophoresis to assess the extent of digestion. Protocols for chromosomal DNA isolation, restriction enzyme digestion, and agarose gel electrophoresis are well known in the art and can be found in many references (e.g., Molecular Cloning: A Laboratory Manual, 2001, Third Edition, Sambrook, J. and Russell, D., Cold Spring Harbor Laboratory Press, Cold Spring Harbor, N.Y.).

Completely digested samples were purified to remove the restriction enzyme and the concentration of digested DNA was determined fluorometrically (PicoGreen® dsDNA Quantitation Kit, Molecular Probes, Eugene, Oreg.). 0.7 μg of purified digested DNA was mixed with competent non-mutant non-resistant B. subtilis DB 170 cells prepared as described in Dubnau, D., and R. Davidoff-Abelson (J. Mol. Biol. Vol. 56, pages 209 to 221 (1971)). The transformation mixture was then incubated at 37° C. for 90 minutes while shaking at 225 rpm. The transformation was then plated onto plates containing rifampicin and incubated for 16-24 hours at 37° C. after which the number of colony forming units (CFU) were determined. The results of the transformation are shown in Table 4.

TABLE 4
B. subtilis Rifampicin Resistance Transformation Data

| | | | | |
|---|---|---|---|---|
| | | | Percent of | |
| | Total No. | Background | Maximum | |
| | rifampicin | Subtracted | Transformation | Transformation |
| Digest | resistant CFU | CFUs | Rate | Effect |

| | | | | |
|---|---|---|---|---|
| AfIIII | 1 | 0 | 1 | Full Effect |
| BbvI | 4 | 3 | 2 | Full Effect |
| BsaAI | 3 | 2 | 2 | Full Effect |
| BsaJI | 0 | 0 | 0 | Full Effect |
| BstYI | 1 | 0 | 1 | Full Effect |
| DdeI | 3 | 2 | 2 | Full Effect |
| Hpy99I | 0 | 0 | 0 | Full Effect |
| MboII | 0 | 0 | 0 | Full Effect |
| NlaIV | 0 | 0 | 0 | Full Effect |
| Sau96I | 4 | 3 | 2 | Full Effect |
| SfcI | 3 | 2 | 2 | Full Effect |
| SspI | 3 | 2 | 2 | Full Effect |
| Tsp45I | 0 | 0 | 0 | Full Effect |
| ApoI | 14 | 13 | 7 | Middle Effect |
| MsII | 7 | 6 | 4 | Middle Effect |
| BsrGI | 38 | 37 | 19 | No Effect |
| BstBI | 23 | 22 | 12 | No Effect |
| Controls: | | | | |
| Background | 1 | — | | |
| Resistant | 200 | 199 | | |
| Parent | | | | |

### Example 2

### Determination of the Location of a Ciprofloxacin Resistance Mutation in H. influenzae

DNA isolation, Restriction Enzyme Digestion, and Transformation

Chromosomal DNA was isolated from H. influenzae strain super 8 (Jane Setlow, Brookhaven National Laboratory) that has a mutation in the gyrA gene that confers resistance to ciprofloxacin. Samples of the purified DNA (1-2 μg) were completely digested with a ten-fold excess of restriction enzyme according to the manufacturer's directions. Portions of the DNA digests were analyzed by agarose gel electrophoresis to assess the extent of digestion. Protocols for chromosomal DNA isolation, restriction enzyme digestion, and agarose gel electrophoresis are well known in the art and can be found in many references (e.g., Molecular Cloning: A Laboratory Manual, 2001, Third Edition, Sambrook, J. and Russell, D., Cold Spring Harbor Laboratory Press, Cold Spring Harbor, N.Y.).

Completely digested samples were purified to remove the restriction enzyme and the concentration of digested DNA was determined fluorometrically (PicoGreen® dsDNA Quantitation Kit, Molecular Probes, Eugene, Oreg.). Two hundred nanograms of purified digested DNA were mixed with competent non-mutant non-resistant H. influenaze NP200 cells prepared as described previously (Barcak, G. J. et al., Methods Enzymol. Vol. 204, pages 321 to 342 (1991)). The transformation mixture was then incubated at 37° C. for 30 minutes. Five ml of supplemented Brain Heart Infusion media (sBHI) was then added, and the cells were incubated at 37° C. for 1 hour. 0.001 ml, 0.01 ml and 0.1 ml aliquots were plated onto sBHI agar plates containing 0.03 μg/ml ciprofloxacin. The plates were incubated overnight at 37° C. to select for growth of resistant colonies. The results of the transformation are shown in Table 5.

TABLE 5

H. influenzae Ciprofloxacin resistance
transformation data

| | | | | |
|---|---|---|---|---|
| | | | Percent of | |
| | Total No. | Background | Maximum | |
| | ciprofloxacin | Subtracted | Transformation | Transformation |
| Digest | resistant CFU | CFUs | Rate | Effect |

| | | | | |
|---|---|---|---|---|
| BbvI | 180 | 0 | 0.0 | Full Effect |
| BsaAI | 180 | 0 | 0.0 | Full Effect |
| BstUI | 300 | 0 | 0.0 | Full Effect |
| Cac8I | 180 | 0 | 0.0 | Full Effect |
| Fnu4HI | 120 | 0 | 0.0 | Full Effect |
| HaeIII | 240 | 0 | 0.0 | Full Effect |
| HhaI | 0 | 0 | 0.0 | Full Effect |
| HinfI | 0 | 0 | 0.0 | Full Effect |
| HphI | 180 | 0 | 0.0 | Full Effect |
| Hpy188I | 300 | 0 | 0.0 | Full Effect |
| HpyCH4IV | 0 | 0 | 0.0 | Full Effect |
| MaeIII | 240 | 0 | 0.0 | Full Effect |
| MboII | 120 | 0 | 0.0 | Full Effect |
| MsII | 300 | 0 | 0.0 | Full Effect |
| NlaIII | 300 | 0 | 0.0 | Full Effect |
| RsaI | 60 | 0 | 0.0 | Full Effect |
| Sau3AI | 0 | 0 | 0.0 | Full Effect |
| SspI | 0 | 0 | 0.0 | Full Effect |
| TaqI | 60 | 0 | 0.0 | Full Effect |
| TfiI | 180 | 0 | 0.0 | Full Effect |
| Tsp45I | 0 | 0 | 0.0 | Full Effect |
| HpyCH4V | 360 | 60 | 0.0 | Full Effect |
| AseI | 1100 | 800 | 0.1 | Full Effect |
| MnII | 1300 | 1000 | 0.1 | Full Effect |
| HpyCH4III | 1600 | 1300 | 0.2 | Full Effect |
| TspRI | 3100 | 2800 | 0.3 | Middle Effect |
| AfIIII | 7400 | 7100 | 0.9 | Middle Effect |
| Hpy99I | 11000 | 10700 | 1.3 | Middle Effect |
| SmII | 23000 | 22700 | 2.8 | No Effect |
| SfcI | 50000 | 49700 | 6.1 | No Effect |
| BstYI | 65000 | 64700 | 7.9 | No Effect |
| AcII | 76000 | 75700 | 9.2 | No Effect |
| DdeI | 83000 | 82700 | 10.1 | No Effect |
| BfaI | 220000 | 219700 | 26.8 | No Effect |
| HpaII | 820000 | 819700 | 100.0 | No Effect |
| Controls: | | | | |
| Background | 300 | — | | |
| Resistant | 600000 | 599700 | | |
| Parent | | | | |

### Example 3

### Summary of Experimental Data and Analysis of for Identification of Five Mutations in H. influenzae

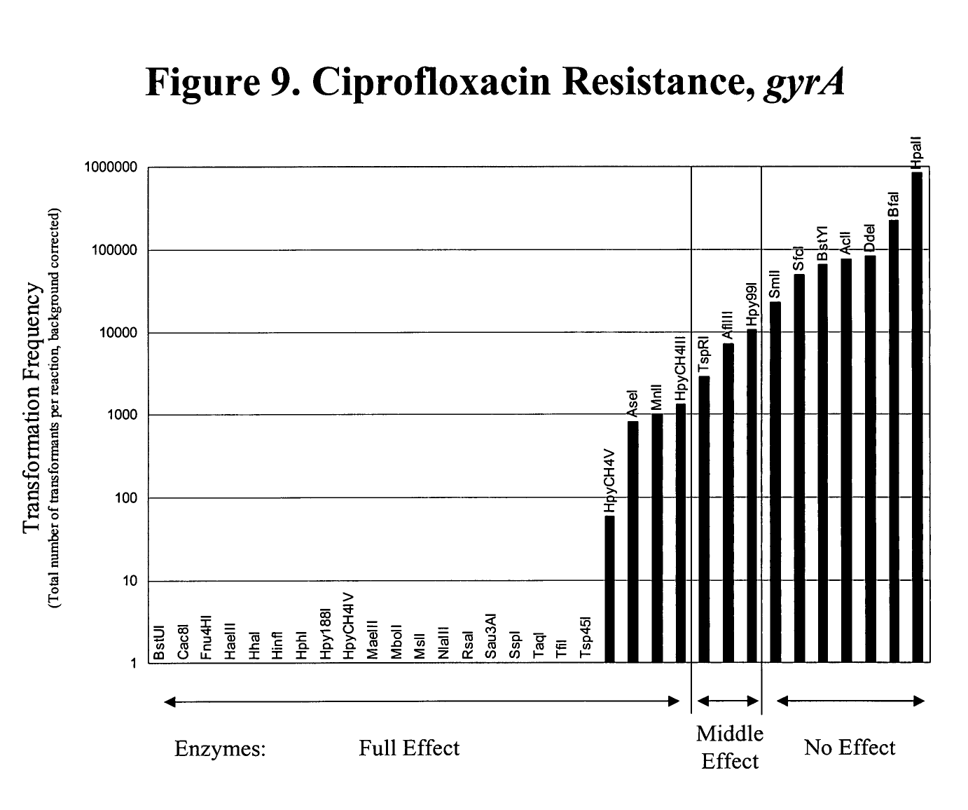

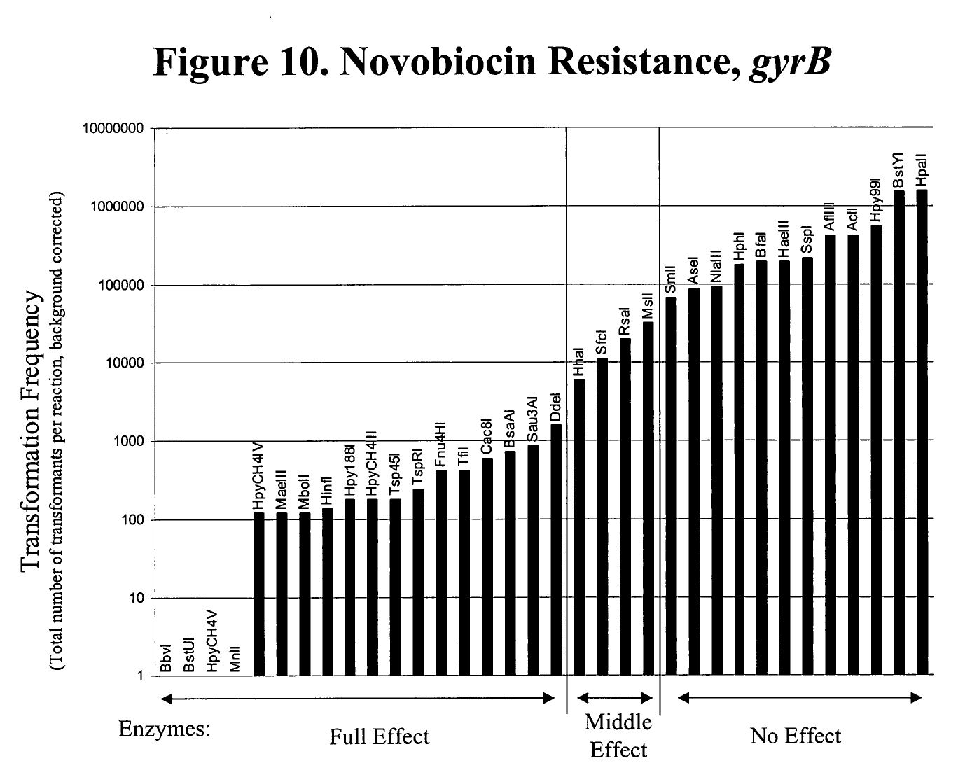

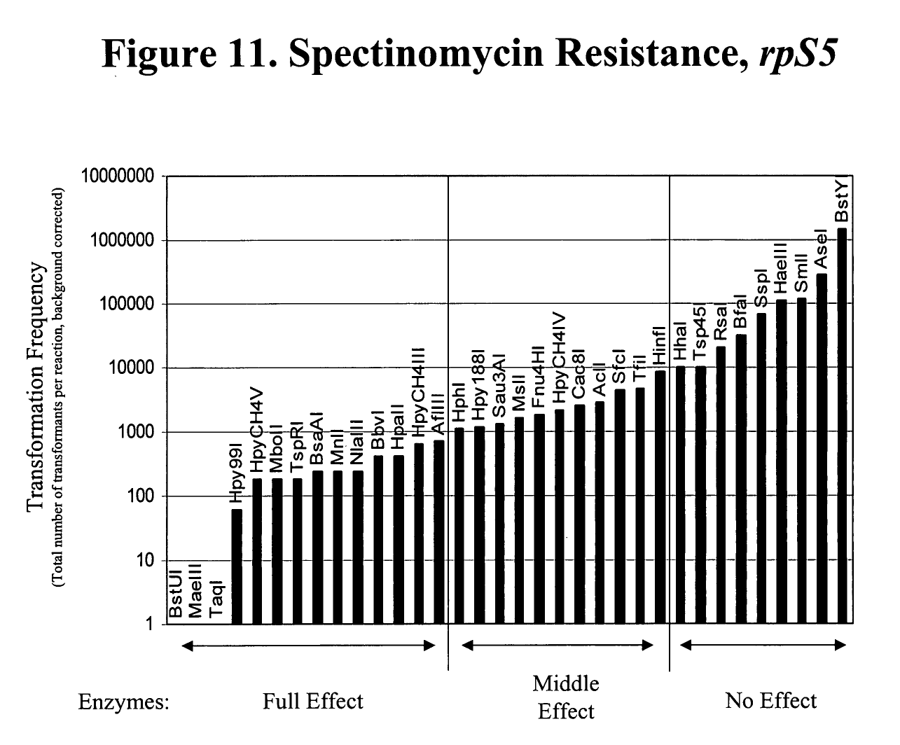

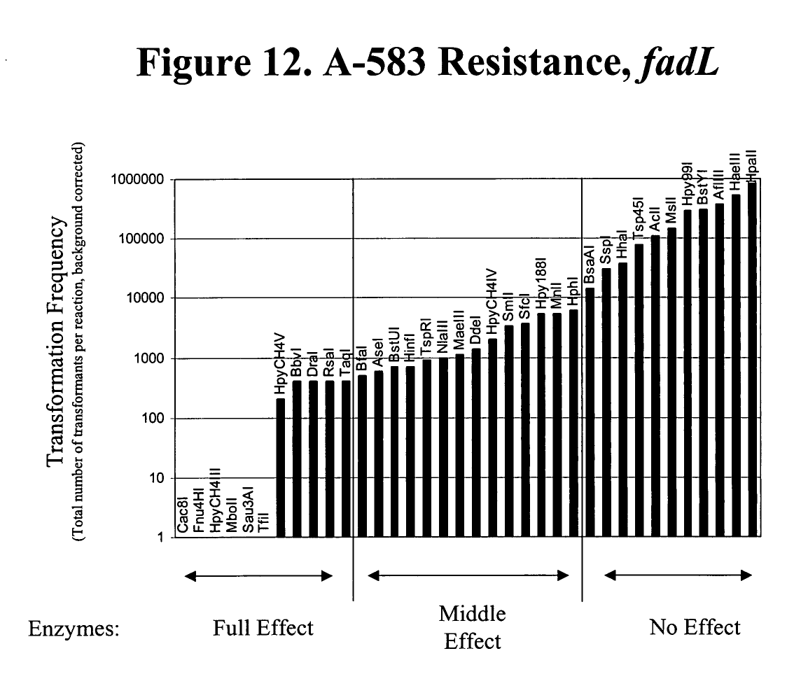

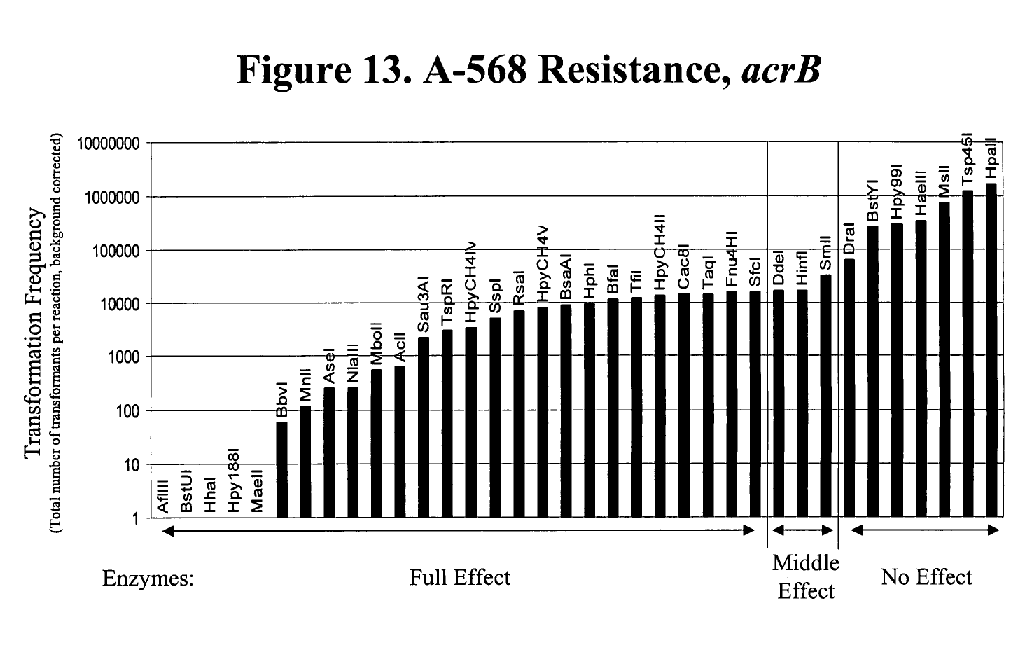

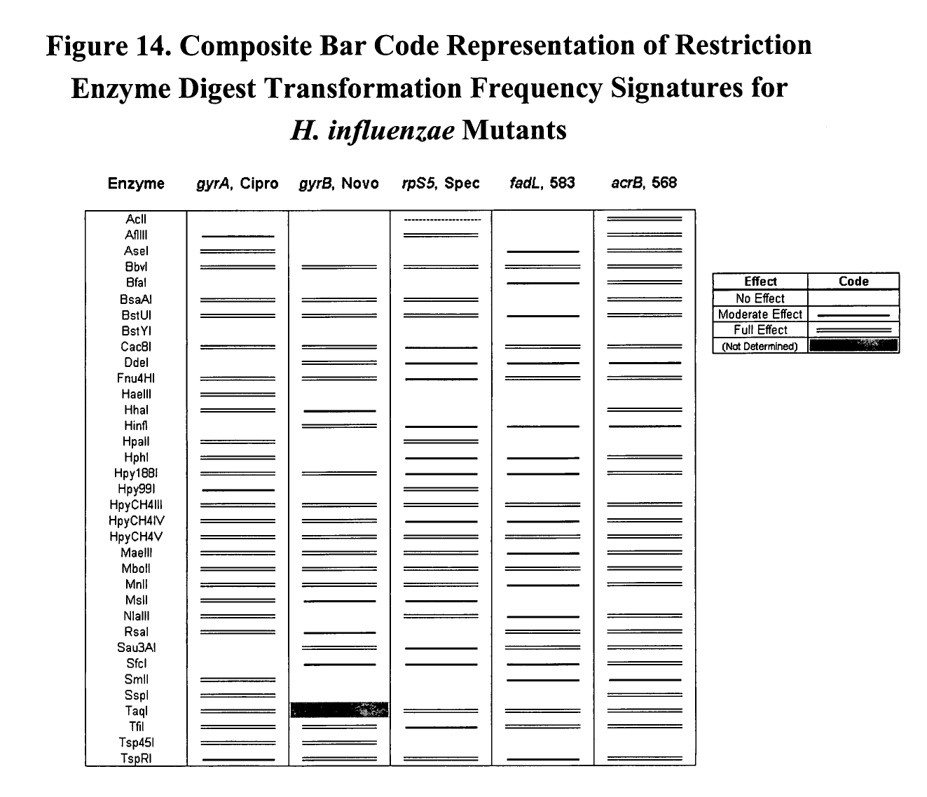

The analysis was performed on four mutants of H. influenzae in addition to the mutant containing the ciprofloxacin resistance mutation in gyrA. Additional resistance mutations were assessed by the method of the invention, a novobiocin resistance mutation in gyrB and a spectinomycin resistance mutation in rpS5. Mutations to antibacterial compounds with unknown mechanisms of action were also analyzed. Resistance to Abbott compound A-583 was found to be due to a mutation resistance in fadL, and resistance to Abbott compound A-568 was found to be due to a mutation in acrB. The data for these analyses are shown in FIGS. 9, 10, 11, 12, and 13 as well as Tables 1 and 2. A bar code representation of the data is also shown in FIG. 14.

Significant differences in the shape of the transformation bar charts, as well as the relative positions of the restriction enzymes, are observed for the different mutants. The difference in transformation patterns is highlighted and summarized for comparison in the composite bar code shown in FIG. 14.

In the case of the spectinomycin resistance mutation in rpS5, for an ≧80% fit between the experimental and calculated data, the analysis had to be run without considering the presence of USS DNA uptake sequences since the mutation was more than 3,000 bp away.

1. A method of identifying the location of a mutation in the genome of a particular organism, said method comprising:
a) isolating DNA from an organism having a mutated phenotype,
b) contacting said DNA with a panel of restriction enzymes to produce several fragments of said DNA,
c) introducing said fragments of DNA into a non-mutated host organism to transform said non-mutated organism into a mutated organism that expresses the mutated phenotype,
d) determining the transformation frequency by counting number of the originally non-mutated host organisms of step (c) that express said mutated phenotype, and
e) correlating said transformation frequency to the known locations of the restriction enzyme sites of step (b), to provide information regarding the location of said mutation in the genome.

2. The method of claim 1 wherein, the organism is selected from the group consisting of bacteria, fungi, yeast, Plasmodia and multicellular organisms.

3. The method of claim 2 wherein the bacteria is selected from the group consisting of bacteria for which the entire genomic DNA has been determined and bacteria for which the genomic DNA has been partially determined.

4. The method of claim 3 wherein the bacteria for which the entire genomic DNA has been determined is selected from the group consisting of: Agrobacterium tumefaciens, Caulobacter crescentus, Listeria monocytogenes, Borrelia burgdorferi, Brucella melitensis, Campylobacter jejuni, Clostridium perfringens, Corynebacterium glutamicum, Escherichia coil, Enterococcus faecalis, Helicobacter pylori, Mycoplasma pneumoniae, Mycoplasma genetalium, Pasteurella multocida, Pseudomonas aeruginosa, Pseudomonas putida, Pseudomonas syringae, Rickettsia prowazekii, Salmonella enterica, Salmonella typhimurium, Staphylococcus aureus, Streptococcus Pneumoniae, Streptococcus pyogenes, Xanthomonas campestris pv. canpestris, Yersinia pestis, Bacillus subtilis, Deinococcus radiodurans, Haemophilus influenzae, Lactococcus lactis, Neisseria meningitidis, Nostoc sp, Streptococcus mutans, Streptomyces coelicolor, and Synechocystis sp.

5. The method of claim 3 wherein the bacteria for which the entire genomic DNA has been partially determined is selected from the group consisting of: Acetobacter xylinum, Acholeplasma laidlawii, Acinetobacter baumannii, Actinobacillus pleuropneumoniae, Actinomyces vvscosus, Agrobacterium rhizogenes, Amycolatopsis mediterranei, Amycolatopsis orientalis, Anabaena spp, Azospirillum brasilense, Azotobacter vinelandii, Bacillus cereus, Bacillus parapertussis, Bacillus thuringiensis, Bacillus licheniformis, Bacillus sphaericus, Bacillus thuringiensis, Bacteroides fragilis, Bordetella pertussis, Bradyhizobium japonicum, Brevibacterium flavum, Brevibacterium lactofermentum, Brucella abortus, Butyrivibrio fbrisolvens, Citrobacter freundii, Clavibacter michiganensis, Clostridium botulinum, Clostridium cellulolyticum, Clostridium difficile, Cyanobacterium chroococcidiopsis, Cytophaga johnsonae, Dichelobacter nodosus, Enterobacter aerogenes, Enterobacter agglomerans, Enterococcus hirae, Erwinia carotovora, Francisella spp, Fremyella diplosiphon, Giardia lambia, Klebsiella pneumoniae, Lactobacillus acidophilus, Lactobacillus casei, Lactobacillus delbrueckii, Lactobacillus fermentum, Lactobacillus gasseri, Lactobacillus helveticus, Lactobacillus plantarum, Lactobacillus salivarius, Lactobacillus teuteri, Legionella pneumophila, Leptospira biflexa, Leuconostoc spp, Methylobacterium extorquens, Mannheimia haemolytica, Methylophillus spp, Mycobacterium aurum, Mycobacterium bovis, Mycobacterium smegmatis, Myxococcus xanthus, Pasteurelia haemolytica, Pasteurella trehalosi, Pediococcus acidilactici, Propionibacterium jensenii, Proteus spp, Pseudomonas oleovorans, Rhizobium leguminosarum, Rhodococcus equi, Rhodopseudomonas viridis, Rhodospirillum molischianum, Rochalimaea quintana, Rubrivivax gelatinosus, Saccharopolyspora erythraea, Salmonella senftenburg, Seratia spp, Serpula hyodysenteriae, Spirulina platensis, Streptococcus cremoris, Streptococcus parasanguis, Streptococcus salivarus, Streptococcus sanguis, Sulfolubus Shibatae, Synechococcus sp., Toxoplasma gondii, Vibrio anguillarum, Vibrio spp, Yersinia pseudotuberculosis, Yersinia enterocolitica and, Zymomonas mobilis.

6. The method of claim 2, wherein the multicellular organism is mammalian.

7. The method of claim 1 wherein, the panel restriction enzymes is selected from the group consisting of AciI, AclI, AfIII, AluI, ApoI, AseI, BbvI, BfaI, BsaAI, BsaHI, BsaJI, BsrFI, BssKI, BstUI, BstYI, Cac8I, DdeI, Dral, FnuHI, FokI, HaeIII, HhaI, HinfI, HpaII, HphI, Hpy188I, Hpy99I, HpyCH4III, HpyCH4IV, HpyCH4V, MaeIII, MboII, MnlI, MseI, MslI, NlaIII, NlaIV, RsaI, Sau3AI, Sau96I, SfaNI, SfcI, SmlI, SspI, TaqI, TfiI, TseI, Tsp45I, Tsp509I, and TspRI.

8. The method of claim 1 wherein said panel of restriction enzymes comprise 10 to 50 of said restriction enzymes.

9. The method of claim 1 wherein said transformation process of step (c) occurs by a process selected from the group comprising transduction and electroporation.

10. The method of claim 1 wherein, the mutated phenotype is selected from the group consisting of drug resistance, increased production of proteins, increased ability to degrade waste and increased ability to detect analytes.

11. The method of claim 10 wherein the increased production of proteins comprises increased production of therapeutically useful biologicals, increased production of industrial enzymes and increased production of secondary metabolites.

12. The method of claim 11 wherein the increased production of secondary metabolites comprises secondary metabolites with pharmacological activities.

13. The method of claim 10 wherein the drug resistance is resistance to antibacterial agents.

14. The method of claim 1 wherein, the correlation of said transformation frequency to the known locations of said restriction enzyme sites is obtained by a restriction map print out.

15. The method of claim 1 wherein, the correlation of said transformation frequency to the known locations of said restriction enzyme sites is obtained by a computerized program.

16. A method of identifying the precise locus and identity of a mutation in the—genome of a particular organism, said method comprising:
a) isolating DNA from an organism having a mutated phenotype,
b) contacting said DNA with a panel of restriction enzymes to produce several fragments of said DNA,
c) introducing said fragments of DNA into a non-mutated host organism to transform said non-mutated organism into a mutated organism that expresses the mutated phenotype,
d) determining the transformation frequency by counting number of the originally non-mutated host organisms of step (c) that express said mutated phenotype,
e) correlating said transformation frequency to the known locations of the restriction enzyme sites of step (b), to provide information regarding the location of said mutation in the genome,
and further comprising,
f) amplifying candidate locations of said mutation in the genome by Polymerase Chain Reaction (PCR) using DNA from the mutant as template,
g) testing the amplified candidate locations for the ability to transform non-mutated host cells,
h) sequencing the amplified candidate location that transforms with high frequency, and
i) comparing the sequence of the amplification product to the sequence of the parent strain to precisely identify the locus and the identity of the mutation in the genome of a particular organism.

17. The method of claim 16 wherein, the organism is selected from the group consisting of bacteria, fungi, yeast, Plasmodia and multicellular organisms.

18. The method of claim 17 wherein the bacteria is selected from the group consisting of bacteria for which the entire genomic DNA has been determined and bacteria for which the genomic DNA has been partially determined.

19. The method of claim 17, wherein the multicellular organism is a mammalian.

20. The method of claim 16 wherein, the panel restriction enzymes is selected from the group consisting of AciI, AclI, AflIII, AluI, ApoI, AseI, BbvI, BfaI, BsaAI, BsaHI, BsaJI, BsrFI, BssKI, BstUI, BstYI, Cac8I, DdeI, DraI, FnuHI, FokI, HaeIII, HhaI, HinfI, HpaII, HphI, Hpy188I, Hpy99I, HpyCH4III, HpyCH4IV, HpyCH4V, MaeIII, MboII, MnlI, MseI, MslI, NlaIII, NlaIV, RsaI, Sau3AI, Sau96I, SfaNI, SfcI, SmlI, SspI, TaqI, TfiI, TseI, Tsp45I, Tsp509I, and TspRI.

21. The method of claim 16 wherein, the mutated phenotype is selected from the group consisting of drug resistance, increased production of proteins, increased ability to degrade waste and increased ability to detect analytes.

22. The method of claim 21 wherein the drug resistance is resistance to antibacterial agents.

23. A computerized method of identifying location of chromosomal mutations in the genome of a particular organism using a computer program, said method comprising:
a) inputting enzyme transformation data into a computer, wherein said enzyme transformation data comprises the results of frequency of transformation of non-mutated host organism after introduction of DNA fragments from a mutated organism, wherein said DNA fragments have been digested by known restriction enzymes,
b) inputting known map of restriction enzyme cleavage sites into said computer,
c) inputting a group of variables that affect frequency of transformation into said computer,
d) correlating inputs of steps (a), (b), and (c) to genome coordinate through said computer program, wherein said computer program scans genome sequence to identify locations of restriction enzyme cleavage sites in the genome that best fit the transformation frequency data, and
e) comparing the transformation frequency data with the genome restriction enzyme cleavage map to identify the location of the mutation.

24. The method of claim 23 wherein the variables of step (c) comprise: (a) the distance of the mutation from the end of the DNA segment, (b) the length of the DNA segment and, and (c) in some species the presence of uptake signal sequences.
